(19) United States
(12) Patent Application Publication
(10) Pub. No.: US 2018/0133713 A1
(43) Pub. Date: May 17, 2018

(54) MICROFLUIDIC IN-VITRO SCREENING CHIP SYSTEM AND METHOD OF USING THE SAME

(71) Applicant: National Tsing Hua University, Hsinchu City (TW)

(72) Inventors: Gwo-Bin Lee, Hsinchu City (TW); Lien-Yu Hung, Hsinchu City (TW); Chih-Hung Wang, Hsinchu City (TW); Chien-Yu Fu, Hsinchu City (TW); Wen-Bin Lee, Hsinchu City (TW)

(73) Assignee: National Tsing Hua University, Hsinchu City (TW)

(21) Appl. No.: 15/431,783

(22) Filed: Feb. 14, 2017

(30) Foreign Application Priority Data
Nov. 17, 2016 (TW) 105137596

(51) Int. Cl. B01L 3/00; G01N 33/543; C12N 15/10

(52) U.S. Cl. CPC B01L 3/502715; G01N 33/54386; C12N 15/1048; C12N 15/1037; B01L 2400/06; B01L 2300/0867; B01L 2200/10; B01L 2200/16; B01L 2300/0819; B01L 2300/0636; B01L 2300/0816

(57) ABSTRACT

A microfluidic in-vitro screening chip system including a first micromixer chamber, a plurality of first storage chambers, a second micromixer chamber and a plurality of second storage chambers. The first micromixer chamber includes a non-target disease tissue slide region. The plurality of first storage chambers are connected to the first micromixer chamber, wherein at least one first storage chamber is used for storing a library. The second micromixer chamber is connected to the first micromixer chamber, and the second micromixer chamber includes a target disease tissue slide region. The plurality of second storage chambers are connected to the second micromixer chamber.

### CROSS-REFERENCE TO RELATED APPLICATION

This application claims the priority benefit of Taiwan application serial no. 105137596, filed on Nov. 17, 2016. The entirety of the above-mentioned patent application is hereby incorporated by reference herein and made a part of this specification.

### BACKGROUND

### Field of the Invention

The invention is directed to a chip system and a method of using the same. More particularly, the invention is directed to a microfluidic in-vitro screening chip system and a method of using the same.

### Description of Related Art

An in-vitro screening system plays an important role in screening of a variety of affinity reagents, specific binding molecules, biomarkers and even in drug screening. Generally speaking, methods which are most often applied to in-vitro screening include systematic evolution of ligands by exponential enrichment (SELEX) and phage display. However, these techniques have to perform sampling, mixing, washing, collecting, redissolving and amplifying target molecules repeatedly. Thus, well-trained researchers are in need for performing repetitive and intensive manual operations with the use of a great number of samples and reagents, which causes difficulty in widely popularizing the applications of these in-vitro screening techniques.

The conventional detection requires too much effort and time, thus, cannot effectively achieve the purpose of fast detection. Currently, an in-vitro screening technique capable of achieving fast detection of target diseases is required.

### SUMMARY

The invention provides a microfluidic in-vitro screening chip system and a method of using the same, capable of quickly screening to select a screening target with high specificity and affinity for a target disease tissue slide, so as to achieve a purpose of fast detection.

A microfluidic in-vitro screening chip system provided by the invention includes a first micromixer chamber, a plurality of first storage chambers, a second micromixer chamber and a plurality of second storage chambers. The first micromixer chamber includes a non-target disease tissue slide region. The first storage chambers are connected to the first micromixer chamber, wherein at least one first storage chamber is used for storing a library. The second micromixer chamber is connected to the first micromixer chamber and includes a target disease tissue slide region. The second storage chambers are connected to the second micromixer chamber.

In an embodiment of the invention, the microfluidic in-vitro screening chip system further includes a fluid control module, wherein the first micromixer chamber, the first storage chambers, the second micromixer chamber and the second storage chambers are connected to one another through the fluid control module.

In an embodiment of the invention, the fluid control module includes a plurality of pipelines and a plurality of pneumatic valves, and the pneumatic valves are located in the pipelines.

In an embodiment of the invention, the fluid control module further includes a plurality of gas chambers. The gas chambers are respectively connected to the pneumatic valves, the first micromixer chamber and the second micromixer chamber.

In an embodiment of the invention, the microfluidic in-vitro screening chip system further includes a gas control layer and a liquid control layer, wherein the first micromixer chamber, the first storage chambers, the second micromixer chamber, the second storage chambers and the fluid control module are located in the gas control layer and the liquid control layer.

In an embodiment of the invention, the microfluidic in-vitro screening chip system further includes a slide fixing layer, wherein the liquid control layer is disposed between the gas control layer and the slide fixing layer.

In an embodiment of the invention, the microfluidic in-vitro screening chip system further includes a non-target disease tissue carrier and a target disease tissue carrier. The non-target disease tissue carrier is fixed in the slide fixing layer and disposed correspondingly to the non-target disease tissue slide region. The target disease tissue carrier is fixed in the slide fixing layer and disposed correspondingly to the target disease tissue slide region.

In an embodiment of the invention, the microfluidic in-vitro screening chip system further includes an adhesive layer disposed between the slide fixing layer and the liquid control layer.

In an embodiment of the invention, the library is a single stranded DNA library or a phage displayed oligopeptide library.

In an embodiment of the invention, the first storage chambers include a library storage chamber and a first washing solution storage chamber. The library storage chamber is used for storing the library and a binding buffer. The first washing solution storage chamber is used for storing a washing solution.

In an embodiment of the invention, the second storage chambers include a second washing solution storage chamber, a waste liquid storage chamber and a buffer storage chamber. The second washing solution storage chamber is used for storing a washing solution. The waste liquid storage chamber is used for storing a waste liquid. The buffer storage chamber is used for storing a binding buffer.

In an embodiment of the invention, the microfluidic in-vitro screening chip system further includes an amplification chamber. The amplification chamber is connected to the second micromixer chamber.

In an embodiment of the invention, the microfluidic in-vitro screening chip system further includes a transporting unit. The transporting unit is connected between the first micromixer chamber and the second micromixer chamber.

A method of using a microfluidic in-vitro screening chip system provided by the invention includes the following steps. Step 1: the microfluidic in-vitro screening chip system as recited above is provided. Step 2: a library is provided to the non-target disease tissue slide region to bind the library onto the non-target disease tissue carrier by performing a binding reaction. Step 3: the non-target disease tissue slide region is washed. Step 4: the library which is unbound is transported from the non-target disease tissue slide region to the target disease tissue slide region. Step 5: the library is bound onto a target disease tissue carrier in the target disease tissue slide region by performing a binding reaction, so as to obtain a screening target bound onto the target disease tissue carrier.

In an embodiment of the invention, the method of using the microfluidic in-vitro screening chip system further includes step 6: washing the target disease tissue slide region to remove the unbound library from the target disease tissue slide region.

In an embodiment of the invention, the method of using the microfluidic in-vitro screening chip system further includes repeating cycles of step 2 to step 6 after a step of amplifying the library.

In an embodiment of the invention, the method of using the microfluidic in-vitro screening chip system further includes a step of amplifying the library bound onto the target disease tissue carrier.

In an embodiment of the invention, the library is a single stranded DNA library, and the screening target is an aptamer.

In an embodiment of the invention, the step of amplifying the library includes amplifying a single stranded DNA in the single stranded DNA library by a polymerase chain reaction (PCR).

In an embodiment of the invention, the library is a phage displayed oligopeptide library, and the screening target is an oligopeptide.

In an embodiment of the invention, the step of performing the amplification on the library includes transporting a cell host, a culture solution and the library to the amplification chamber, and making a phage in the phage displayed oligopeptide library invade into the cell host for the amplification.

In an embodiment of the invention, a shear stress of a fluid flowing in the first micromixer chamber and the second micromixer chamber in the microfluidic in-vitro screening chip system is controlled within a range between 0.1 nN and 400 nN.

To sum up, in the microfluidic in-vitro screening chip system and the method using the same provided by the invention, a non-screening target can be excluded by binding of the non-screening target to the non-target disease tissue slide (which is referred to as negative selection), and the screening target can be obtained by binding of the screening target to the target disease tissue slide (which is referred to as positive selection). Thereby, the screening target with high specificity and affinity can be selected by screening with a single-chip system, so as to achieve the purpose of fast detection of target diseases.

In order to make the aforementioned and other features and advantages of the invention more comprehensible, several embodiments accompanied with figures are described in detail below.

### BRIEF DESCRIPTION OF THE DRAWINGS

The invention contains at least one color photograph. Copies of the disclosure publication with the color photographs will be provided by the Patent & Trademark Office upon request and payment of the necessary fee. The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic top view of a microfluidic in-vitro screening chip system according to an embodiment of the invention.

FIG. 2 is an exploded view of the microfluidic in-vitro screening chip system according to an embodiment of the invention.

FIG. 3 is a flowchart of a method of using the microfluidic in-vitro screening chip system according to an embodiment of the invention.

FIG. 4 is a fluorescence signal comparison diagram of a normal tissue slide and cancer tissue slides which were stained with fluorescence by using an aptamer.

FIG. 5 is a fluorescence signal comparison diagram of a normal tissue slide and cancer tissue slides which were stained with fluorescence by using an oligopeptide.

### DESCRIPTION OF EMBODIMENTS

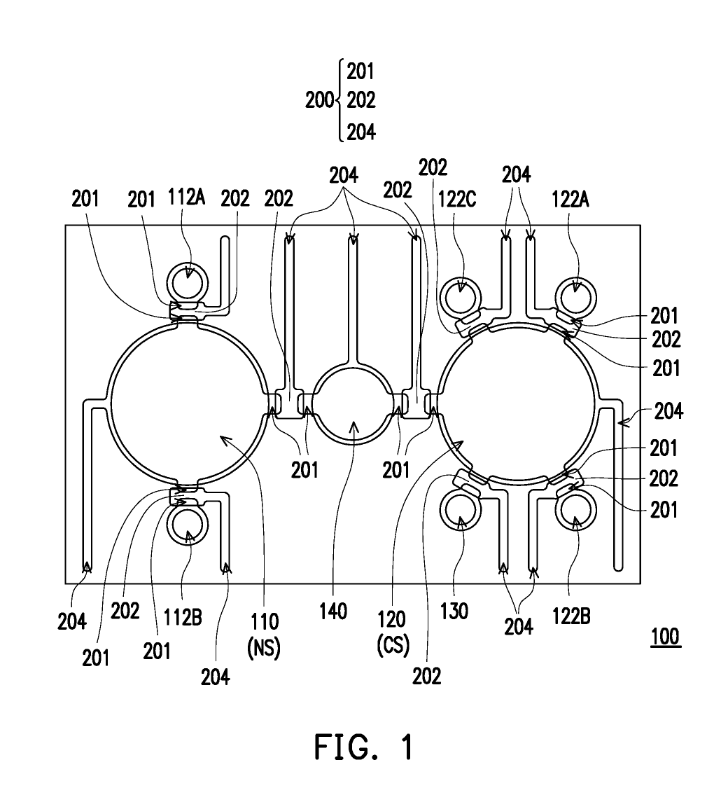

FIG. 1 is a schematic top view of a microfluidic in-vitro screening chip system according to an embodiment of the invention.

Referring to FIG. 1, a microfluidic in-vitro screening chip system 100 in an embodiment of the invention includes a first micromixer chamber 110, a plurality of first storage chambers (112A and 112B), a second micromixer chamber 120 and a plurality of second storage chambers (122A, 122B and 122C) and may further include an amplification chamber 130.

The first micromixer chamber 110 includes a non-target disease tissue slide region NS. The first storage chambers (112A and 112B) are connected to the first micromixer chamber 110. In the present embodiment, a non-target disease tissue slide is, for example, a normal tissue slide. However, the invention is not limited thereto, and any tissue slide of a non-target disease may be used as the non-target disease tissue slide in the invention.

In the present embodiment, the first storage chambers include a library storage chamber 112A and a first washing solution storage chamber 112B. The library storage chamber 112A is used for storing a library and a binding buffer. The library is, for example, a single stranded DNA library or a phage displayed oligopeptide library. The first washing solution storage chamber 112B is used for storing a washing solution. In the present embodiment, the first storage chambers are described as including the first library storage chamber 112A and the first washing solution storage chamber 112B for example; however, a person with ordinary skill in the art may adjust the number and storage objects of the first storage chambers based on demands, and it pertains to the scope to be protected by the invention as long as at least one first storage chamber is used for storing the library.

In addition, the second micromixer chamber 120 is connected to the first micromixer chamber 110. The second micromixer chamber 120 includes a target disease tissue slide region CS. In the present embodiment, a target disease tissue slide is, for example, a cancer tissue slide. However, the invention is not limited thereto, and the person with ordinary skill in the art may adjust the choice of target disease tissue slide according to the type of target disease to be detected.

The second storage chambers (122A, 122B and 122C) are connected to the second micromixer chamber 120, and the amplification chamber 130 is connected to the second micromixer chamber 120. In the present embodiment, the second storage chambers include a second washing solution storage chamber 122A, a waste liquid storage chamber 122B and a buffer storage chamber 122C. The second washing solution storage chamber 122A is used for storing a washing solution. The waste liquid storage chamber 122B is used for storing a waste liquid. The buffer storage chamber 122C is used for storing a binding buffer. In the present embodiment, the second storage chambers are described as including the second washing solution storage chamber 122A, the waste liquid storage chamber 122B and the buffer storage chamber 122C for example; however, the person with ordinary skill in the art may adjust the number and storage objects of the second storage chambers based on demands.

In the present embodiment, the microfluidic in-vitro screening chip system 100 further includes a fluid control module 200. The first micromixer chamber 110, the first storage chambers (112A and 112B), the second micromixer chamber 120 and the second storage chambers (122A, 122B and 122C) are connected to one another through the fluid control module 200. To be specific, the fluid control module 200 includes a plurality of pipelines 201 and a plurality of pneumatic valves 202. The pipelines 201 are respectively connected to the first micromixer chamber 110, the first storage chambers (112A and 112B), the second micromixer chamber 120 and the second storage chambers (122A, 122B and 122C). Additionally, the pneumatic valves 202 are located in the pipelines 201. When the pneumatic valves 202 are opened, the first micromixer chamber 110, the first storage chambers (112A and 112B), the second micromixer chamber 120 and the second storage chambers (122A, 122B and 122C) may communicate with one another. That is, the communication state among the first micromixer chamber 110, the first storage chambers (112A and 112B), the second micromixer chamber 120 and the second storage chambers (122A, 122B and 122C) is controlled by the pneumatic valves 20 to be opened or closed.

For instance, one of the pipelines 201 and one of the pneumatic valves 202 are located between the library storage chamber 112A and the first micromixer chamber 110. When the pneumatic valve 202 between the library storage chamber 112A and the first micromixer chamber 110 is opened, a fluid (i.e., a library) may be transported from the library storage chamber 112A to the first micromixer chamber 110 through the pipeline 201.

Additionally, the fluid control module 200 may further include a plurality of gas chambers 204. The gas chambers 204 are respectively connected to the pneumatic valves 202, the first micromixer chamber 110 and the second micromixer chamber 120. Whether the pneumatic valves 202 are opened or closed and the manner of the fluid flowing in each chamber are controlled by a positive pressure or a negative pressure generated by each of the gas chamber 204.

In addition, referring to the embodiment illustrated in FIG. 1, the microfluidic in-vitro screening chip system 100 includes a transporting unit 140. The transporting unit 140 is connected between the first micromixer chamber 110 and the second micromixer chamber 120. When the fluid is transported from the first micromixer chamber 110 to the second micromixer chamber 120, the transporting unit 140 may be temporarily used as a storage chamber. However, the invention is not limited thereto. For instance, in another embodiment, the microfluidic in-vitro screening chip system 100 may exclude the transporting unit 140, and the first micromixer chamber 110 is connected to the second micromixer chamber 120 only through the pipelines 201. Additionally, the manner of the fluid flowing in the transporting unit 140 may be controlled by the fluid control module 200.

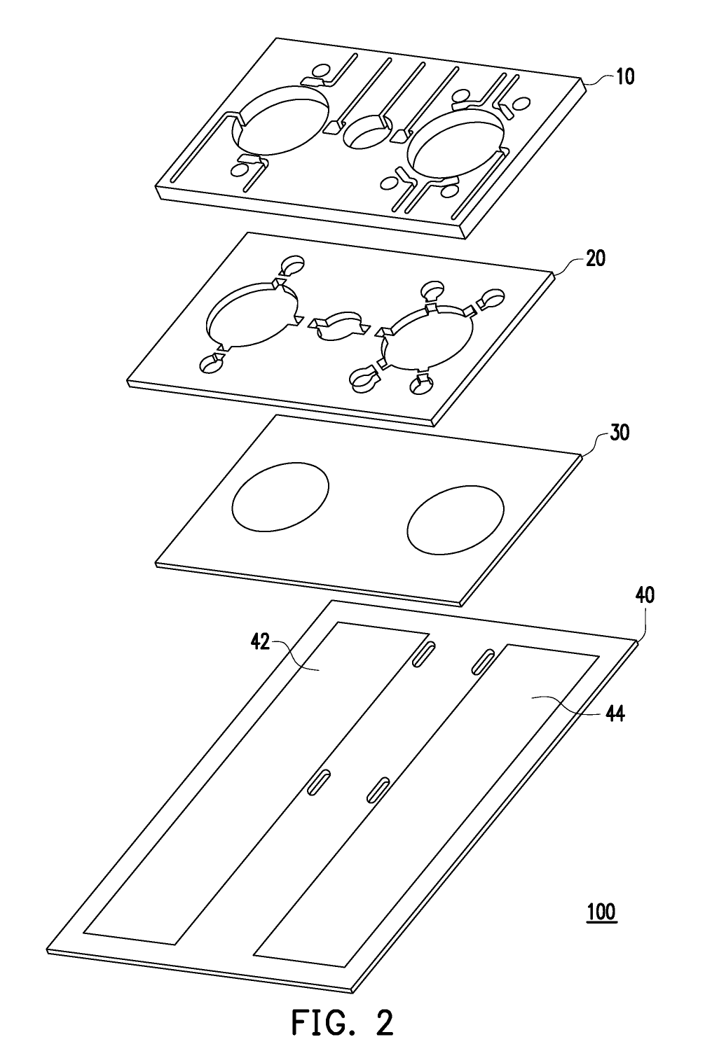

FIG. 2 is an exploded view of the microfluidic in-vitro screening chip system according to an embodiment of the invention.

Next, referring to FIG. 2, the microfluidic in-vitro screening chip system 100 further includes a gas control layer 10 and a liquid control layer 20. In the present embodiment, the first micromixer chamber 110, the first storage chambers (112A and 112B), the second micromixer chamber 120, the second storage chambers (122A, 122B and 122C) and the fluid control module 200 are located in the gas control layer 10 and the liquid control layer 20. A material of the gas control layer 10 and the liquid control layer 20 includes polydimethylsiloxane.

Additionally, the microfluidic in-vitro screening chip system 100 further includes a slide fixing layer 40. The liquid control layer 20 is disposed between the gas control layer 10 and the slide fixing layer 40. A material of the slide fixing layer 40 includes acrylics.

In the present embodiment, the microfluidic in-vitro screening chip system 100 further includes a non-target disease tissue carrier 42 and a target disease tissue carrier 44. The non-target disease tissue carrier 42 is fixed in the slide fixing layer 40 and disposed correspondingly to the non-target disease tissue slide region NS. In addition, the target disease tissue carrier 44 is fixed in the slide fixing layer 40 and disposed correspondingly to the target disease tissue slide region CS.

To be specific, the non-target disease tissue carrier 42 is used for carrying a non-target disease tissue slide, so as to provide the non-target disease tissue slide to the non-target disease tissue slide region NS in the first micromixer chamber 110. In the same way, the target disease tissue carrier 44 is used for carrying a tissue slide with a target disease (which is a cancer tissue slide, for example), so as to provide the target disease tissue slide to the target disease tissue slide region CS in the second micromixer chamber 120. It should be noted that a size of the slide fixing layer 40 is greater than sizes of the gas control layer 10 and the liquid control layer 20 in the present embodiment, but the invention is not limited thereto. For instance, the size of the slide fixing layer 40 may be adjusted according to the sizes of the non-target disease tissue carrier 42 and the target disease tissue carrier 44, so as to achieve a preferable size.

Referring back to FIG. 2, the microfluidic in-vitro screening chip system 100 further includes an adhesive layer 30 disposed between the slide fixing layer 40 and the liquid control layer 20. In the present embodiment, the adhesive layer 30 is, for example, a double-sided tape adhesive layer, but the invention is not limited thereto, as long as the adhesive layer 30 is a film layer capable of achieving an adhesion effect. In another embodiment, the person with ordinary skill in the art may also achieve the adhesion between the slide fixing layer 40 and the liquid control layer 20 by using a surface modification method. Accordingly, the gas control layer 10, the liquid control layer 20, adhesive layer 30 and the slide fixing layer 40 may be integrated to form the structure of the microfluidic in-vitro screening chip system 100 of the embodiment of the invention.

According to embodiments described above, the microfluidic in-vitro screening chip system 100 includes the first micromixer chamber 110 with the non-target disease tissue slide region NS and the second micromixer chamber 120 with the target disease tissue slide region CS. Thereby, the non-screening target may be excluded by binding of the non-screening target to the non-target disease tissue slide (which is referred to as negative selection), and a screening target may be selected by binding of the screening target to the target disease tissue slide (which is referred to as positive selection). Thereby, the screening target with high specificity and affinity may be selected by screening with a single-chip system, so as to achieve the purpose of fast detection of target diseases.

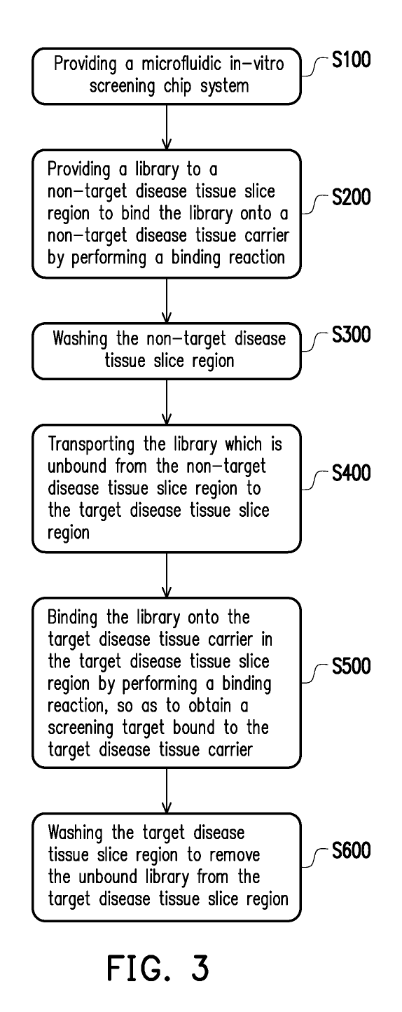

Hereinafter, a method of using the microfluidic in-vitro screening chip system 100 will be described with reference to FIG. 3.

FIG. 3 is a flowchart of a method of using the microfluidic in-vitro screening chip system according to an embodiment of the invention.

Referring to both FIG. 1 and FIG. 3, step S100 is performed, where the microfluidic in-vitro screening chip system 100 is provided. The structure of microfluidic in-vitro screening chip system 100 may be referred to the descriptions with reference to FIG. 1 and FIG. 2.

Then, step S200 is performed, where a library is provided to the non-target disease tissue slide region NS to bind the library onto the non-target disease tissue carrier 42 by a binding reaction. The library may be a single stranded DNA library or a phage displayed oligopeptide library. The library which is mixed with a binding buffer may be placed in the library storage chamber 112A and transported to the non-target disease tissue slide region NS in the first micromixer chamber 110 through the fluid control module 200. When the library is transported to the non-target disease tissue slide region NS, a portion of the library is bound to the non-target disease tissue slide carried on the non-target disease tissue carrier 42 by performing a binding reaction. In other words, the library having high affinity with the non-target disease tissue is bound to the non-target disease tissue carrier 42. In the present embodiment, the time for the binding reaction is not particularly limited, and the time may range from 1 minute to 60 minutes, which is preferably 30 minutes.

Thereafter, step S300 is performed, where the non-target disease tissue slide region NS is washed. In this step, the non-target disease tissue slide region NS is washed by the washing solution transported from the first washing solution storage chamber 112B to the non-target disease tissue slide region NS in the first micromixer chamber 110 through the fluid control module 200. This step may be used for washing and separating the library which is unbound to the non-target disease tissue on the non-target disease tissue carrier 42.

Next, step S400 is performed, where the unbound library is transported from the non-target disease tissue slide region NS to the target disease tissue slide region CS. In this step, the unbound library may first be temporarily stored in the transporting unit 140, and after the unbound library is completed washed off, the library is then transported from the transporting unit 140 to the target disease tissue slide region CS in the second micromixer chamber 120. In another embodiment, in case where the microfluidic in-vitro screening chip system 100 is not provided with the transporting unit 140, the unbound library may be directly transported to the target disease tissue slide region CS in the second micromixer chamber 120.

Then, step S500 is performed, where the library is bound to the target disease tissue carrier 44 in the target disease tissue slide region CS by performing a binding reaction, so as to obtain a screening target bound onto the target disease tissue carrier 44. In this step, the binding reaction is performed by using the fluid control module 200 to transport the binding buffer from the buffer storage chamber 122C to the target disease tissue slide region CS in the second micromixer chamber 120. The library having high affinity or specificity with the target disease tissue is bound to the target disease tissue of the target disease tissue slide, and the library which is unbound or has low affinity is removed. Additionally, in the present embodiment, a time for the binding reaction with the target disease tissue slide is not particularly limited, and the time may range from 1 minute to 60 minutes, which is preferably 30 minutes.

Then, step S600 is performed, where the target disease tissue slide region CS is washed to remove the unbound library from the target disease tissue slide region CS. To be more specific, the step of washing the target disease tissue slide region CS is performed by the washing solution transported from the second washing solution storage chamber 122A to the target disease tissue slide region CS in the second micromixer chamber 120 through the fluid control module 200. In the present embodiment, the washing step may be performed for several times to remove the library with less binding capability. Thereby, the library which is unbound or has low affinity is removed through the washing step, and is treated as a waste liquid and transported to the waste liquid storage chamber 122B. Additionally, the library bound to the target disease tissue of the target disease tissue slide has higher binding force or specificity with the target disease tissue, hence may be used as the screening target.

In addition, in the method of using the microfluidic in-vitro screening chip system, step S200 to step S600 may further be repeated in cycles, so as to obtain the screening target with higher affinity. As for the number of cycles, it may be adjusted based on experimental requirements.

In the embodiment described above, the method of using the microfluidic in-vitro screening chip system further includes a step of amplifying the library bound in the target disease tissue slide region CS. To be specific, the library bound in the target disease tissue slide region CS may be amplified after step S600. Additionally, step S200 to step S600 may also be repeated in cycles after the step of amplification.

In the embodiment described above, when the library in use is a single stranded DNA library, the screening target is an aptamer. More particularly, the step of amplification may be performed on the single stranded DNA library bound in the target disease tissue slide region CS. The step of amplification includes amplifying a single stranded DNA in the single stranded DNA library by a polymerase chain reaction (PCR). For instance, the amplification chamber 130 of the present embodiment may be used as a PCR chamber. Thus, the single stranded DNA library bound in the target disease tissue slide region CS may be transported to the amplification chamber 130 for PCR. In this way, cloning and gene sequencing may be performed on the selected single stranded DNA to obtain the screening target for the aptamer.

In the embodiment described above, when the library in use is a phage displayed oligopeptide library, the screening target is an oligopeptide. More particularly, the step of amplification may be performed on the phage displayed oligopeptide library bound in the target disease tissue slide region CS. The step of amplification includes transporting the single stranded DNA library bound in the target disease tissue slide region CS, a cell host and a culture solution to the amplification chamber 130, such that a phage in the phage displayed oligopeptide library invades into the cell host for the amplification. To be more specific, the cell host may be, for example, Escherichia coli (E. coli), and the phage invades into the bacterial cytoplasm for replication and is then released from the E. coli. In this way, cloning and gene sequencing may be performed on the selected phage to obtain the screening target of the oligopeptide.

In the method of using the microfluidic in-vitro screening chip system described above, two screening methods, including systematic evolution of ligands by exponential enrichment (SELEX) and phage display, may be used in a single microfluidic system. Additionally, a shear stress of the fluid (e.g., the library) flowing in the first micromixer chamber 110 and the second micromixer chamber 120 in the microfluidic in-vitro screening chip system 100 may be controlled within a range between 0.1 nN and 400 nN, so as to enhance the screening effect. Moreover, the screening is performed by using the target disease tissue slide in the present embodiment, which is different from the conventional method where the screening is performed only by using target disease cell lines cultivated in laboratories. Furthermore, an aptamer obtained from the cultivated cell lines still needs to go through multiple tests before its application in clinical diseases. By contrast, in the present invention, a disease tissue slide may be directly used for screening, and an aptamer with specificity obtained in that way may be directly applied in a clinical disease tissue slide of the same type. Accordingly, the screening target (e.g., the aptamer or the oligopeptide) obtained through screening by using the microfluidic in-vitro screening chip system 100 of the present embodiment may be directly used in clinical detection.

### EXPERIMENTAL EXAMPLES

Experimental examples provided below can prove that the microfluidic in-vitro screening chip system 100 described in the embodiments above may be used for screening to select a screening target with high affinity and specificity for a target disease tissue. In addition, examples with respect to the screening of an aptamer and an oligopeptide of a cancer will be exemplarily described in the experimental examples below. However, it should be noted that the target disease tissue referred to in the invention is not limited to cancer and may be applicable to any target disease tissue to be screened.

### Experimental Example 1

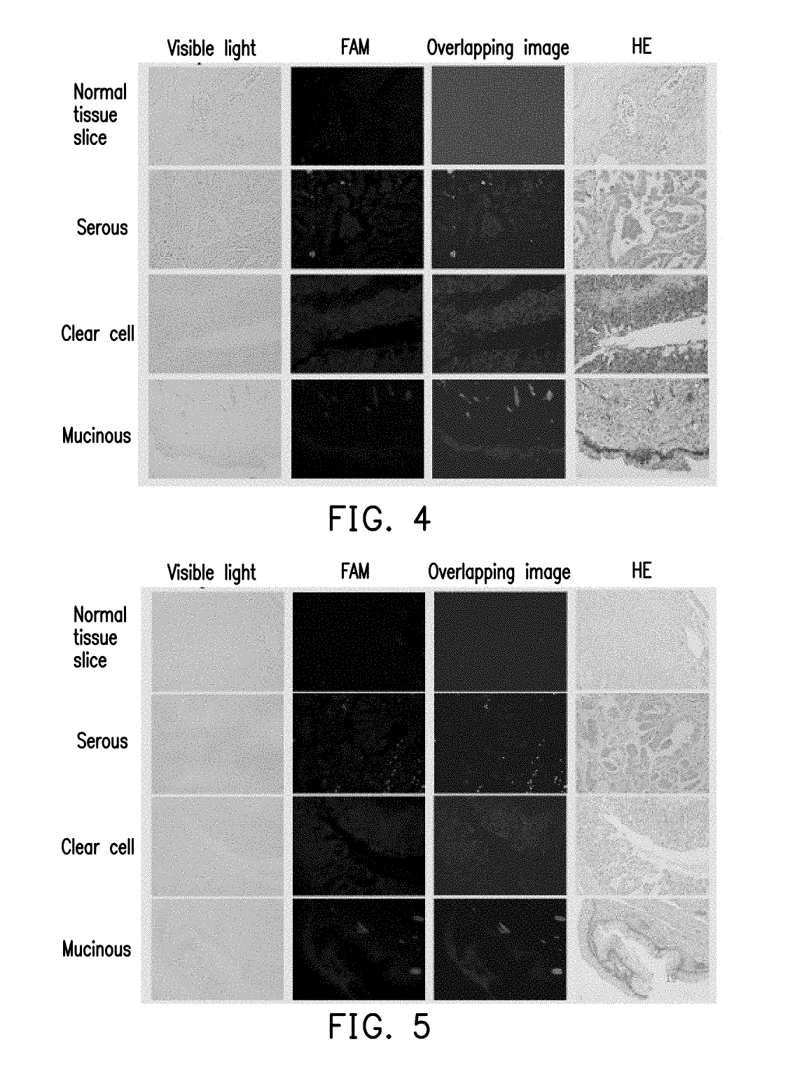

In Experimental example 1, a single stranded DNA library was used as a library, and a tissue slide from a patient with ovarian cancer was selected and used as a target disease tissue slide. Types of ovarian cancer include serous carcinoma, clear cell carcinoma and mucinous carcinoma. After the microfluidic in-vitro screening chip system 100 depicted in FIG. 1 was used, and the method of using the microfluidic in-vitro screening chip system depicted in FIG. 3 was performed, a sequence of an aptamer treated as a screening target was obtained. After the sequence was obtained, the aptamer was synthesized by a chemical method and a 5′-end of the aptamer was modified with carboxyfluorescein (FAM), and the FAM generated a green fluorescence signal as being excited by blue light. Next, the modified aptamer was respectively mixed with a normal tissue slide (used as a non-target disease tissue slide) and various types of ovarian cancer tissue slides (including a serous, a clear cell and a mucinous ovarian cancer tissue slides) for fluorescence staining, and fluorescence signals were observed. The experiment results are illustrated in FIG. 4.

### Experimental Example 2

In Experimental example 2, a phage displayed oligopeptide library was used as a library, and a tissue slide from a patient with ovarian cancer was selected and used as a target disease tissue slide. Types of ovarian cancer include serous carcinoma, clear cell carcinoma and mucinous carcinoma. After the microfluidic in-vitro screening chip system 100 depicted in FIG. 1 was used, and the method of using the microfluidic in-vitro screening chip system depicted in FIG. 3 was performed, a sequence of an oligopeptide treated as a screening target was obtained. After the sequence was obtained, the oligopeptide was synthesized by a chemical method, and an N-terminus of the oligopeptide was modified with FAM. Then, the modified oligopeptide was respectively mixed with a normal tissue slide (used as a non-target disease tissue slide) and various types of ovarian cancer tissue slides (including a serous, a clear cell and a mucinous ovarian cancer tissue slides) for fluorescence staining, and fluorescence signals were observed. The experiment results are illustrated in FIG. 5.

FIG. 4 is a fluorescence signal comparison diagram of a normal tissue slide and cancer tissue slides which were stained with fluorescence by using an aptamer. FIG. 5 is a fluorescence signal comparison diagram of a normal tissue slide and cancer tissue slides which were stained with fluorescence by using an oligopeptide.

FIG. 4 and FIG. 5 respectively illustrate visible light images, FAM fluorescence images and overlapping images of the visible light images and the FAM fluorescence images of the normal tissue slide and various types of ovarian cancer tissue slides which were stained with fluorescence. The experiment results show that, under the observation by a fluorescence microscope, obvious green fluorescence signals may be observed in both the selected aptamer and oligopeptide in the cancer tissue slides. By contrast, no obvious green fluorescence signals may be observed in both the screened aptamer and oligopeptide in the normal tissue slide. In addition, FIG. 4 and FIG. 5 further illustrate images of the normal tissue slide and various types of ovarian cancer tissue slides which were stained by performing a hematoxylin-eosin staining (HE) method. Specific locations of the aptamer and the oligopeptide bound in the ovarian cancer tissue slide may be determined according to the overlapping images of the visible light images and the FAM fluorescence images with the images obtained by performing the HE method. Accordingly, it is evident that the microfluidic in-vitro screening chip system 100 can be used to screen the aptamer and the oligopeptide with high affinity and specificity for cancer tissues.

Based on the above, in the microfluidic in-vitro screening chip system and the method of using the same provided by the embodiments described above, the non-screening target can be excluded by binding of the non-screening target to the non-target disease tissue slide (which is referred to as negative selection), and the screening target can be obtained by binding of the screening target to the target disease tissue slide (which is referred to as positive selection). Thereby, the screening target with high specificity and affinity can be selected by screening with a single-chip system, so as to achieve the purpose of fast detection of target diseases.

Although the invention has been described with reference to the above embodiments, it will be apparent to one of the ordinary skill in the art that modifications to the described embodiment may be made without departing from the spirit of the invention. Accordingly, the scope of the invention will be defined by the attached claims not by the above detailed descriptions.

What is claimed is:

1. A microfluidic in-vitro screening chip system, comprising:
a first micromixer chamber, comprising a non-target disease tissue slide region;
a plurality of first storage chambers, connected to the first micromixer chamber, wherein at least one first storage chamber is used for storing a library;
a second micromixer chamber, connected to the first micromixer chamber and comprising a target disease tissue slide region; and
a plurality of second storage chambers, connected to the second micromixer chamber.

2. The microfluidic in-vitro screening chip system according to claim 1, further comprising a fluid control module, wherein the first micromixer chamber, the first storage chambers, the second micromixer chamber and the second storage chambers are connected to one another through the fluid control module.

3. The microfluidic in-vitro screening chip system according to claim 2, wherein the fluid control module comprises a plurality of pipelines and a plurality of pneumatic valves, and the pneumatic valves are located in the pipelines.

4. The microfluidic in-vitro screening chip system according to claim 3, wherein the fluid control module further comprises a plurality of gas chambers respectively connected to the pneumatic valves, the first micromixer chamber and the second micromixer chamber.

5. The microfluidic in-vitro screening chip system according to claim 2, further comprising a gas control layer and a liquid control layer, wherein the first micromixer chamber, the first storage chambers, the second micromixer chamber, the second storage chambers and the fluid control module are located in the gas control layer and the liquid control layer.

6. The microfluidic in-vitro screening chip system according to claim 5, further comprising a slide fixing layer, wherein the liquid control layer is disposed between the gas control layer and the slide fixing layer.

7. The microfluidic in-vitro screening chip system according to claim 6, further comprising:
a non-target disease tissue carrier, fixed in the slide fixing layer and disposed correspondingly to the non-target disease tissue slide region; and
a target disease tissue carrier, fixed in the slide fixing layer and disposed correspondingly to the target disease tissue slide region.

8. The microfluidic in-vitro screening chip system according to claim 6, further comprising an adhesive layer disposed between the slide fixing layer and the liquid control layer.

9. The microfluidic in-vitro screening chip system according to claim 1, wherein the library is a single stranded DNA library or a phage displayed oligopeptide library.

10. The microfluidic in-vitro screening chip system according to claim 1, wherein the first storage chambers comprise:
a library storage chamber, used for storing the library and a binding buffer; and
a first washing solution storage chamber, used for storing a washing solution.

11. The microfluidic in-vitro screening chip system according to claim 1, wherein the second storage chambers comprise:
a second washing solution storage chamber, used for storing a washing solution;
a waste liquid storage chamber, used for storing a waste liquid; and
a buffer storage chamber, used for storing a binding buffer.

12. The microfluidic in-vitro screening chip system according to claim 1, further comprising an amplification chamber connected to the second micromixer chamber.

13. The microfluidic in-vitro screening chip system according to claim 1, further comprising a transporting unit connected between the first micromixer chamber and the second micromixer chamber.

14. A method of using a microfluidic in-vitro screening chip system, comprising:
step 1: providing the microfluidic in-vitro screening chip system as recited in claim 1;
step 2: providing a library to the non-target disease tissue slide region to bind the library to a non-target disease tissue carrier by performing a binding reaction;
step 3: washing the non-target disease tissue slide region;
step 4: transporting the library which is unbound from the non-target disease tissue slide region to the target disease tissue slide region; and
step 5: binding the library to a target disease tissue carrier in the target disease tissue slide region by performing a binding reaction, so as to obtain a screening target bound to the target disease tissue carrier.

15. The method of using the microfluidic in-vitro screening chip system according to claim 14, further comprising:
step 6: washing the target disease tissue slide region to remove the unbound library from the target disease tissue slide region.

16. The method of using the microfluidic in-vitro screening chip system according to claim 15, further comprising:
repeating cycles of step 2 to step 6 after a step of amplifying the library.

17. The method of using the microfluidic in-vitro screening chip system according to claim 14, further comprising a step of amplifying the library bound onto the target disease tissue carrier.

18. The method of using the microfluidic in-vitro screening chip system according to claim 17, wherein the library is a single stranded DNA library, and the screening target is an aptamer.

19. The method of using the microfluidic in-vitro screening chip system according to claim 18, wherein the step of amplifying the library comprises amplifying a single stranded DNA in the single stranded DNA library by a polymerase chain reaction.

20. The method of using the microfluidic in-vitro screening chip system according to claim 17, wherein the library is a phage displayed oligopeptide library, and the screening target is an oligopeptide.

21. The method of using the microfluidic in-vitro screening chip system according to claim 20, wherein the step of amplifying the library comprises:
transporting a cell host, a culture solution and the library to the amplification chamber, and making a phage in the phage displayed oligopeptide library invade into the cell host for the amplification.

22. The method of using the microfluidic in-vitro screening chip system according to claim 14, wherein a shear stress of a fluid flowing in the first micromixer chamber and the second micromixer chamber of the microfluidic in-vitro screening chip system is controlled within a range between 0.1 nN and 400 nN.
